(19) United States
(12) Patent Application Publication
(10) Pub. No.: US 2022/0117878 A1
(43) Pub. Date: Apr. 21, 2022

(54) SENSORY ENHANCED AND MOISTURIZING HAND SANITIZER FORMULATIONS UTILIZING NON-EMULSION TECHNOLOGIES

(71) Applicant: HOLLYFRONTIER LSP BRAND STRATEGIES LLC, Dallas, TX (US)

(72) Inventors: Tyler McIntyre, Seven Fields, PA (US); Benjamin Green, Petrolia, PA (US); Karissa Mooney, Media, PA (US); Clayton Rozic, Greenbackville, VA (US); Fredeon Valianpour, Amsterdam (NL)

(73) Assignee: HOLLYFRONTIER LSP BRAND STRATEGIES LLC, Dallas, TX (US)

(21) Appl. No.: 17/502,172

(22) Filed: Oct. 15, 2021

Related U.S. Application Data
(60) Provisional application No. 63/092,553, filed on Oct. 16, 2020.

(51) Int. Cl. A61K 8/92; A61K 8/34; A61Q 17/00

(52) U.S. Cl. CPC A61K 8/92; A61K 8/345; A61K 2800/30; A61Q 17/005; A61K 8/34

(57) ABSTRACT

Hand sanitizer formulations described herein include sensory enhancing and/or moisturizing ingredients but lack emulsification systems. Exemplary hand sanitizer formulations include low molecular weight alkanes/mineral oils, high emollient loadings which build viscosity and allow for shake-and-use products, and density equalization technology that allows for robust delivery of homogenous product in a shake-and-use formulation.

This application claims priority to U.S. Provisional Patent Application Ser. No. 63/092,553, entitled “Sensory Enhanced and Moisturizing Hand Sanitizer Formulations Utilizing Non-Emulsion Technologies,” filed Oct. 16, 2020, the entire contents of which are hereby incorporated by reference.

The present disclosure relates to hand sanitizer formulations which include sensory enhancing and/or moisturizing ingredients but lack emulsification systems.

Public health events such as COVID-19 have highlighted the need for effective hand sanitizer formulations that are fit for everyday use. Repeated use of alcohol-based hand sanitizers and repeated hand washing can cause dry skin through removal of natural skin oils/fats. Many traditional hand sanitizer formulations are limited in their ability to deliver sensory-enhancing and/or moisturizing ingredients. For example, low levels of aloe vera extract or glycerin can be added to alcohol-based hand sanitizer. In order to utilize a broader range of emollients, traditionally complex emulsions systems are used, which have challenges of large ingredient lists and necessitate the use of emulsion forming equipment.

### SUMMARY

The present disclosure pertains to alcohol-based hand sanitizer formulations that include sensory enhancing and/or moisturizing ingredients and lack emulsification systems, as well as methods for their preparation.

Inclusion of sensory enhancing and/or moisturizing components in an alcohol-based hand sanitizer formulation is achieved in several ways including the use of low molecular weight alkanes/mineral oils, the use of high emollient loadings which build viscosity and allow for shake-and-use products, and the use of density equalization technology that allows for robust delivery of homogenous product in a shake-and-use formulation.

Preferred embodiments of the hand sanitizer formulations described herein allow consumers to experience a range of delivered sensory and skin health benefits. Formulations range from light, spray-applied formulations for everyday use, to high emollient loading formulations that can coat and protect skin through delivery of barrier forming ingredients such as petrolatum, USP.

### DETAILED DESCRIPTION OF PREFERRED EMBODIMENTS

The present disclosure relates to alcohol-based hand sanitizer formulations that include sensory enhancing and/or moisturizing ingredients but lack emulsification systems. The emollients delivered through this approach have inherent sensory enhancing and/or moisturizing benefits (such as hydrocarbon alkanes, white mineral oil, petrolatum, and vegetable based emollients).

In preferred embodiments of the hand sanitizer formulations described herein, USP grade isopropanol and USP grade ethanol provide the sanitizing functions. Water is also present in most of the inventions as a diluent, density adjuster, and to slow the evaporation rate of the formulation. All preferred embodiments of the hand sanitizer formulations lack emulsifying components.

In preferred embodiments, the minimum concentration of isopropanol present in the formulation is about 70% by volume and the minimum concentration of ethanol present in the formulation is about 60% by volume.

One preferred embodiment of the hand sanitizer formulations disclosed herein comprises low molecular weight alkanes or mineral oils that are suitable for personal care applications. As used herein, “low molecular weight” refers to alkanes of Carbon chain length predominantly below C-22, and centered more closely in distribution from C-12 to C-17 average Carbon chain length. Given these average Carbon chain lengths, the average molecular weight in preferred embodiments could range from approximately 172 g/mol to approximately 240 g/mol. Exemplary low molecular weight alkanes, such as INCI C12-C17 Alkane and INCI C14-C22 Alkane, are soluble in select alcohol and water solutions. Clear, fully solubilized solutions are formed, indicating the complete lack of an emulsion. These fully soluble embodiments are able to incorporate various levels of alkanes, but typically less than 5% by mass. These same embodiments show solubility that is not inhibited by the addition of common alcohol soluble components such as glycerin and hydrogen peroxide. In these fully soluble embodiments, the selection of the alkane and the matching with the appropriate alcohol/water solution is critical. For example, 3% (by volume) of INCI C14-C22 Alkane is soluble into a base solution of 89:11 solution of isopropanol-water, but this same loading of INCI C14-C22 Alkane formed turbidity in a solution of 80.5:19.5 isopropanol-water. In these embodiments, loading of alcohol-soluble fragrances do not interfere with the solubility properties and may enhance the consumer's product experience.

Preferred embodiments of the hand sanitizer formulations in which a low molecular weight alkane or mineral oil, such as saturated hydrocarbon alkane or low viscosity mineral oil, is completely dissolved in an alcohol solution comprise isopropanol at about 70% to about 90% by volume, or in other preferred embodiments about 79% to about 87% by volume, distilled water at about 10% to about 29% by volume, or in other preferred embodiments about 10% to about 19% by volume, and a saturated hydrocarbon alkane or low viscosity mineral oil, where “low viscosity” is less than 5 cSt viscosity at 40° C., at about 0.25% to about 5% by volume, or in other preferred embodiments about 2% to about 3% by volume. In preferred embodiments, the alkane or emollient is a saturated hydrocarbon alkane having a carbon distribution centered near C12 to C17 average carbon chain length. In preferred embodiments, the saturated hydrocarbon alkane may be C12-C17 alkane or C14-C22 alkane. Optionally, preferred embodiments of the hand sanitizer formulations may further comprise about 1.4% to about 1.5% glycerin by volume and may also further comprise about 1% fragrance by volume. In these preferred embodiments, the saturated hydrocarbon alkane or low viscosity mineral oil should be included at less than 10% by weight of the hand sanitizer formulation.

An additional preferred embodiment of the hand sanitizer formulations disclosed herein comprises high emollient loadings. Emollients can deliver excellent sensory profiles at high loadings (ex. 30% emollient). These levels far exceed those that are typically delivered via emulsion technology (ex. 3% emollient). Certain preferred embodiments can even reach a 30% loading of Petrolatum, USP which is sufficient to be labeled as a skin protectant. Use of this type of sanitizer could significantly counter the drying effects experienced by repeated hand washing that is common during public health events. Public health workers, doctors, and others that necessitate severe hand washing protocols would be well served by these high loadings of emollient. High emollient loading hand sanitizers utilize solid, semi-solid, and liquid emollients. Their incorporation into the alcohol base causes a dramatic increase in the viscosity (relative to alcohol) which leads to semi-inclusion of the alcohol into the semi-solid matrix. Formulations may undergo slight separation, but the embodiments can be homogenized through brief agitation by the consumer. Consumer agitation is acceptable for use in other areas of personal health and personal care (for example, two-phased mouthwash and two-phased makeup remover, among others). Because of the high viscosity of the emollients used in this embodiment of the invention, a significant amount of the sanitizing alcohol will remain entrapped in the emollient structure. Re-homogenization can be aided by packaging design if necessary or desired (ex. ball bearings). Alcohol or oil soluble fragrances may be used to enhance the consumer experience with the product.

Preferred embodiments of the hand sanitizer formulations in which emollients are added to an alcohol solution comprise isopropanol at about 70% by volume and one or more emollients at about 30% by volume, where the emollient may be petrolatum, such as sensory enhanced petrolatum, mineral oil, an oil or wax of a vegetable, or an oil or wax of synthetic origin. In preferred embodiments, the emollient may include one or more isoparaffins and synthetic or natural alkanes, synthetic and petroleum based waxes (including but not limited to Fischer Tropsch (FT), linear alpha olefin waxes, slack waxes, footsoil waxes, microcrystalline waxes) beeswax, silicone oils, plant based oils of soybean, jojoba, sunflower, safflower, rapeseed, avocado, shea, olive, coconut, castor, canola, and other triglyceride based oil products. The emollient should be present at greater than 10% by weight. Optionally, preferred embodiments of the hand sanitizer formulations may further comprise about 1% fragrance by volume.

Additional preferred embodiments of the hand sanitizer formulations described herein utilize precise pairing of the density of the alcohol/water base to the suspended emollient. The density of common alcohol (isopropanol/ethanol) solutions (including water) are in the range of emollients that are manufactured to precise densities. When, by volume, isopropanol is greater than 70%, or ethanol greater than 60%, the solutions are commonly known to be effective as hand sanitizer. When mineral oil, or other precisely controlled emollients such as alkanes, are added to these density matched alcohol solutions, the droplets of oil (which are not soluble in the hydrophilic solution) will have a greatly reduced tendency to sink or float in the alcohol base. Light agitation can disperse these droplets for long durations. An example of a preferred formulation is prepared by adding 3% of Density Equivalent Mineral Oil (matched to the IPA/water solution by volume) to a base of isopropanol/water at 80.5/19.5 ratio (by volume). In this example, the isopropanol/water density at 20° C. is approximately 0.846 g/cc. The mineral oil is also precisely manufactured to this same approximate density. As a result, after initial agitation this suspension will remain dispersed for far longer than a common consumer interaction with the product (less than 30 seconds). Properly paired dispersions, such as the aforementioned embodiment may display stability after “shake” agitation ranging from approximately 10 minutes to well over 6 hours depending on the conditions of the sample and formulation. The density equalization also increases the efficacy of basic agitation, protecting against consumers who do not follow product direction as strictly as desired. Embodiments with emollients that are precisely paired to density of the alcohol base show easy resuspension with basic actions such as 3-4 inversions of the sanitizer container. Similar agitation actions on density-mismatched products (which do not use the technology described in this disclosure) will quickly separate once agitation ceases and increases the chance that consumers would sample non-homogenous product during product dispensing. Small loadings of alcohol soluble fragrances are able to be incorporated and may require additional density measurements for each fragrance/loading combination. These fragrances will enhance the consumer experience with the product.

Additional preferred embodiments of the hand sanitizer formulations comprise emollients added to an alcohol solution at a loading of less than 10% by weight wherein the emollient is paired precisely to the density of the alcohol/water solution. Preferred embodiments of these hand sanitizer formulations may comprise an alcohol solution that comprises isopropanol or ethanol at about 74% to about 77% by volume, or about 69% to about 73% by weight, and distilled water at about 20% to about 24% by volume, or about 24% to about 28% by weight, and one or more emollients at about 2.3% to about 2.8% by volume, or about 2.4% to about 2.9% by weight, where the emollient may be a density equalized white mineral oil having a target of 0.82 g/mL to 0.89 g/mL at 20° C. The emollient should be present at less than 10% by weight with a targeted density difference not more than 2%. Optionally, preferred embodiments of the hand sanitizer formulations may further comprise about 1% fragrance by volume.

In all embodiments disclosed herein, other non-emulsifying ingredients can be incorporated in order to enhance the sensory appeal, or efficacy of the final product. Fragrances and dyes are groupings of ingredients that the aforementioned embodiments may utilize without interfering with the physical and chemical phenomenon that are exhibited.

Some ingredients will be classified as water or alcohol soluble and include pH adjusters (that may be used for the facilitation of thickeners) and extracts for fragrance or sensory/claim enhancement (example, aloe, lemon extract, glycerin). Thickeners such as carbomer-based or acrylate-based thickeners may be used to drive the viscosity of the fully soluble alkane formulations or the density equalized formulations to a gel or lotions like consistency. Others non-emulsifying ingredients may be oil soluble and be included in the emollient package. These ingredients include but are not limited to isoparaffins and synthetic or natural alkanes, synthetic and petroleum based waxes (including but not limited to FT, linear alpha olefin waxes, slack waxes, footsoil waxes, microcrystalline waxes) beeswax, silicone oils, plant based oils of soybean, jojoba, sunflower, safflower, rapeseed, avocado, shea, olive, coconut, castor, canola, and other triglyceride based oil products. Oil and alkane products from natural and synthetic routes can also be used in each area of this invention, including but not limited to alkanes of vegetable or triglyceride origin and oils derived from catalytic or solvent based dewaxing processes.

Embodiments of the invention may utilize packaging technology that aids the dispensing and use of the product with or without consumer agitation. Examples of such technology include the use of ball bearing and other shear-promoting devise. Further examples include pressurized containers, multi-compartment packaging and dual dispensing containers.

Certain preferred embodiments of hand sanitizer formulations disclosed herein utilize the unique solubility characteristics of certain alkane products in suitable alcohol bases. When blended with specific alcohol/water combinations, turbidity free solutions are formed, and no emulsion is present. The alkanes used in this approach are unique due to their low molecular weight and their complex molecular structure. Alkanes used in this type of embodiment generally show full solubility in the 0.5-5% range in alcohol solutions that are tailored to their properties. Isopropanol (USP grade) is especially effective in the production of full solubility sanitizer products based on alkanes such as INCI C12-C17 Alkane and INCI C14-C22 Alkane. The aforementioned alkanes are comprised of highly saturated (>99% saturates) hydrocarbons with linear, isoparaffinic, and cyclic hydrocarbons structures with tightly controlled molecular weight profiles. These alkanes show total insolubility in water. The carbon chain length of the alkanes used in the preferred embodiments are approximately, but not limited to, C10-C26 in chain length. The properties of molecular weight and carbon structure lead to solubility in specific alcohol solutions, some of which fall in the range of effective sanitizers (>70% isopropanol). When added to hand sanitizer formulations, the characteristic moisture retention and sensory enhancing properties will be imparted to the skin. Emollients of mineral oil origin (for example, mineral oil of less than 5 cSt viscosity at 40° C., with specific gravity of approximately 0.818 at 25° C./25° C.), show solubility similar to alkane products and can be used at approximate loading of 3% in a solution of 80.5:19.5 isopropanol-water. The simplest embodiment disclosed herein may be formed by combining a sanitizing alcohol, an alkane product, and water. Certain preferred formulations of the hand sanitizer formulations are disclosed in Table 1 below.

TABLE 1

| Ingredient | Exemplary Hand Sanitizer Formulation | | | | | |
|---|---|---|---|---|---|---|
| (Volume %) | 1 | 2 | 3 | 4 | 5 | 6 |
| Isopropanol (USP | 78.9% | 86.8% | 80.5% | 85% | 79.7% | 86.0% |
| Grade) | | | | | | |
| Water (Distilled) | 19.1% | 10.1% | 16.05% | 10.5% | 15.9% | 10.0% |
| INCI C12-C17 Alkane | 2% | — | 2% | — | 2% | — |
| INCI C14-C22 Alkane | — | 3% | — | 3% | — | 3.0% |
| Glycerin, USP | — | — | 1.45% | 1.5% | 1.4% | — |
| Fragrance | — | — | — | — | 1.0% | 1.0% |

Exemplary formulations 1 and 2 shown in Table 1 above can be modified and enhanced in a myriad of ways. Specific emollients of mineral oil origin are also fully soluble in Isopropanol/water solutions at specific ratios. For example, in most cases, a fragrance can improve the user experience with the product. In other cases, the use of alcohol soluble ingredients such as glycerin can be added to lower the total volatility of the formulation

Additional preferred embodiments of the hand sanitizer formulations are created when high levels of emollient are used. These emollient loadings tend to drive the viscosity of the formulation far above that of formulations which do not use high emollient loadings or thickeners. These high emollient loadings provide a significant level of sensory modification and moisturization when added to suitable alcohol bases (namely, USP Isopropyl alcohol and USP ethanol). In certain examples, the level of Petrolatum, USP can be taken to the threshold at which it can be viewed as a “Skin Protectant” (30% minimum). These embodiments display sensory properties that are similar to some lotions or creams. Due to the intense level of emollients in these formulations, their applications would typically differ from lower emollient loading formulations. For example, high emollient sanitizers could be used upon return from a trip during which repeated hand washing was necessary, as it is known that repeated hand washing can leave skin devoid of natural barrier properties. The core emollients used for these embodiments are comprised of Petrolatum, vegetable oils, vegetable waxes, mineral oil, alkanes, and a myriad of other synthetic and natural emollients. These emollients have low solubility in sanitizing alcohol bases (USP ethanol>60% and USP Isopropanol>70%). Effective formulations are proposed that are easily re-homogenized by consumer agitation. There is precedence for consumer agitation as witnessed in two-phased mouthwash formulations and cosmetic formulations such as makeup remover. Re-homogenization can be aided by the use of specialized packaging (for example, ball bearings which increase shear within the package during consumer agitation). The simplest form of these embodiments includes the combination of Isopropyl alcohol (USP grade) and a sensory-enhanced petrolatum, and a natural emollient such as hydrogenated castor oil. The White Petrolatum, USP used in this embodiment is sensory enhanced and designed to have ultra-low drag and easy spreadability on the consumers' skin. Sensory enhanced petrolatum is comprised of petroleum hydrocarbons including waxy and oil-based streams. Petrolatums prepared for sensory enhanced applications include those that are described in U.S. Patent Publication No. US 2010/0189674 A1, incorporated by reference herein. As such the petrolatums have one or more of the following physical properties: (a) a flow onset of about 45° C. or less, (b) a storage modulus at 0.1 Pa (G′=0.1 Pa) of about 50° C. or less, and (c) a complex viscosity at 0.1 Pas (m=0.1 Pa's) of about 45° C. or less. In addition, fragrance can be added in order to enhance the customer experience. Certain preferred formulations of the hand sanitizer formulations are disclosed in Table 2 below.

TABLE 2

| | Exemplary Hand Sanitizer Formulation | | | | | |
|---|---|---|---|---|---|---|
| Ingredient | 1 | | 2 | | 3 | |
| Isopropanol (USP | 68.5% | (w/w) | 68.5% | (w/w) | 70% | (w/w) |
| Grade) | 70.1% | (v/v) | 70.1% | (v/v) | 71.6% | (v/v) |
| Sensory Enhanced | 30% | (w/w) | 30% | (w/w) | 20% | (w/w) |
| Petrolatum, USP | 28.6% | (v/v) | 28.6% | (v/v) | 19.1% | (v/v) |
| CAS # 8009-03-08 | | | | | | |
| Hydrogenated Castor | 1.5% | (w/w) | 1.0% | (w/w) | — | |
| Oil | 1.3% | (v/v) | 0.9% | (v/v) | | |

| INCI C12-C17 Alkane | — | — | 5.0% | (w/w) |
|---|---|---|---|---|
| | | | 5.0% | (v/v) |
| INCI Vegetable oil | — | — | 5.0% | (w/w) |

| | |
|---|---|
| 4.3% | (v/v) |

| | | | | |
|---|---|---|---|---|
| Fragrance | — | 0.5% | (w/w) | — |

| | |
|---|---|
| 0.4% | (v/v) |

Formulation #3 in Table 2 above is an example of a high emollient loading sanitizer formulation that can incorporate more complex non-emulsified emollient packages. In each example more than 15% of hydrophobic emollients are combined into sanitizing alcohol. The resultant products are suitable for shake and use products which will provide a high level of sensory enhancement and moisturization. In certain embodiments the formulations may be packaged in 2 oz. mist-style spray bottles with metal ball bearings for agitation assistance.

Additional preferred embodiments of the hand sanitizer formulations utilize precise matching of density between the sanitizing alcohol base (Ethanol, USP or Isopropanol, USP) and the suspended emollient. The resultant dispersion yields long lasting homogeneity, which will give the consumer ample opportunity to dispense the product. In the absence of a strong physical force pulling the droplets upwards or downwards, they will remain suspended far longer than rudimentary, unpaired base/emollient products. The simplest embodiment of this invention is seen in combinations of alcohol, water, and mineral oil. In preferred embodiments, the hand sanitizer formulation includes an alcohol solution of isopropanol or ethanol at about 60% to about 90% of the hand sanitizer formulation by volume, or about 74% to about 77% by volume, and distilled water at about 10% to about 30% of the hand sanitizer formulation by volume, or about 20% to about 24% by volume. The hand sanitizer formulation may also include about 2.3% to about 2.9% by volume of one or more emollients, where the emollients are less than 10% by weight of the hand sanitizer formulation. The alcohol and water solutions are prepared first followed by the emollient. The targeted density difference between the alcohol/water (for example, ethanol/water) and the suspended emollient is not more than 2%. Both ethanol and isopropanol are suitable bases. Certain preferred formulations of the hand sanitizer formulations are disclosed in Table 3 below.

TABLE 3

| | Exemplary Hand Sanitizer Formulation | | |
|---|---|---|---|
| Ingredient | | 1 | 2 |

| Ethanol (USP Grade) | 69.3% | (w/w) | — |
|---|---|---|---|
| | 74% | (v/v) | |

| | | | |
|---|---|---|---|
| Isopropanol (USP Grade) | — | 72.8% | (w/w) |

| | | | 77% | (v/v) |
|---|---|---|---|---|
| Density Equalized White Mineral Oil | 2.4% | (w/w) | — | |
| (Target 0.845 g/mL @ 20° C.) | 2.3% | (v/v) | | |

| | | | |
|---|---|---|---|
| Density Equalized White Mineral Oil | — | 2.9% | (w/w) |

| | | | | |
|---|---|---|---|---|
| (Target 0.865 g/mL @ 20° C.) | | | 2.8% | (v/v) |
| Water, Distilled | 28.3% | (w/w) | 24.3% | (w/w) |
| | 23.7% | (v/v) | 20.3% | (v/v) |

Preferred embodiments of the hand sanitizer formulations that include density paired emollients and sanitizing bases can also incorporate fragrance components provided the density and solubility characteristics are carefully measured for their effect on density equalization. In the aforementioned embodiments, special care is taken to control the density of the emollient, which may be mineral oil, alkanes, or petrolatum. Fluctuations in density due to processing steps are minimized to create a narrow density specification. For example, one iteration of Density Equalized White Mineral Oil has a density target of 0.865 @ 20° C. with variance not greater than 0.5% from that value. This represents the approximate value of a 77.2/22.8 (v/v) solution of ethanol and water. Similarly, another Density Equalized White Mineral Oil has a density target of 0.845 @ 20° C. with a variance not greater than 0.5% from the value (this represents the approximate value of a 80.5/19.5 (v/v) solution of isopropanol and water).

Exemplary hand sanitizer formulations, such as those from Table 1, can be prepared in the following general manner:
(1) Isopropanol and distilled water should be combined in a primary mixing vessel. Manual or automated agitation is then applied.
(2) Emollient (which may be comprised of C12-C17 Alkane or C14-C22 Alkane), Glycerin (if present), and fragrance (if present) are combined in a secondary mixing vessel.
(3) The contents of the primary and secondary mixing vessels are then combined and agitation should be applied until no layers, films, swirls are present.
4.) Product should be packaged in suitable dispensing container (such as a mist spray bottle).

Exemplary petrolatum and natural emollient formulations, such as those from Table 2, can be prepared using the following general steps:
(1) Petrolatum and other emollients should be added to a primary mixing vessel.
(2) After mixing, Petrolatum and other emollients should be heated to temperatures above their respective melting points (until liquid and translucent).
(3) The mixed Petrolatum and other emollients should be continuously agitated in the primary vessel.
(4) Isopropanol and fragrance are then slowly added to the primary mixing vessel while agitation continues.
(5) Agitation continues until product is near room temperature.
(6) At room temperature the product can be packaged in suitable application vessel. The product can also be infused into a fibrous substrate (such as a “wipe”) and incorporated into a sealed or resealable package.

Exemplary hand sanitizer formulations, such as those from Table 3, can be prepared in the following general manner:
(1) Alcohol (ethanol or isopropanol) and water should be measured by volume and combined in a primary mixing vessel.
(2) Alcohol and water after mixing should be agitated until homogenous and clear.
(3) Mineral oil and other emollients should then be added to the primary mixing vessel and agitated aggressively.
(4) The product should be immediately dispensed into final packaging, such as mist style spray bottles.

### Example 1

The exemplary hand sanitizer formulation #4 from Table 1 was prepared and evaluated as described below.

First, isopropanol (80.5%) was added, according to volume to a primary mixing vessel. Second, distilled water (10.5%) was added, according to volume, to the primary mixing vessel. The vessel was agitated mechanically. Glycerin (1.5% by volume), and emollient (3.0% by volume, comprised of C14-C22 Alkane) was added to a secondary mixing vessel. The components of the secondary mixing vessel were agitated and added to the primary mixing vessel (containing isopropanol and water). Next, the main mixing vessel was agitated mechanically until the solution was homogenous, transparent, and free of swirls and films of unincorporated components. The product was then dispensed into suitable “mist” pump spray bottles or other suitable packaging. The product was found to have a light viscosity that easily dispersed into a mist when dispensed from the mist pump bottles. Spray was applied to one hand (2-3 pumps) and the resulting liquid was rubbed on the entirety of both hands. The product showed steady evaporation. Hands were left with a sensory experience of a light and non-greasy emollient. The dry-hands feeling was not experienced.

The exemplary hand sanitizer formulation #2 from Table 2 was prepared and evaluated as described below.

First, the components listed in Table 2 were converted to weight % to allow for mass-based formulation. Sensory enhanced Petrolatum, Vegetable Oil, C12-C17 Alkane, and a primary mixing vessel (glass) were conditioned to 180° F. Then, the Sensory enhanced petrolatum (20.0% w/w), Vegetable Oil (5.0% w/w), and C12-C17 Alkane (5.0% w/w) were added to the primary mixing vessel. Next, agitation was commenced in the primary mixing vessel via mechanical agitation. Isopropanol, USP (70.0% w/w) was then slowly incorporated into the emollient base in the primary mixing vessel. Agitation commenced until the primary mixing vessel arrived near room temperature. The product was then dispensed into an application vessel or application substrate. For the application vessel, a single ball bearing of size 5/32″ (chrome steel) was placed into the metal pump bottle before sealing. The ball bearing was incorporated to quickly rehomogenize any phase separation that may have occurred. The ball bearing could be felt and heard during agitation (3-4 shaking actions or inversions). The product was observed to be smooth and of higher viscosity than standard non-gelled hand sanitizer formulations. Emollient properties were observed to be silky and smooth and created a much longer playtime than standard hand sanitizer formulations. The emollient was observed to coat the user's skin. A fiber substrate (in this case, a “wipe”) was also saturated with the sanitizer formulation and sealed to prevent alcohol evaporation. Wipes prepared in this fashion showed easy application of the described formulation, and left hands with a noticeable loading of moisturizing and sensory enhancing emollient.

The exemplary hand sanitizer formulation #2 from Table 3 was prepared and evaluated in the following manner.

First, isopropanol, USP (77% v/v) was added to the primary mixing vessel (a separatory funnel of approx. 2 L). Then, distilled water (20.3% v/v) was added to the primary mixing vessel. The primary mixing vessel was sealed and agitated mechanically until water and isopropanol were homogenous. Next, mineral oil (2.8% v/v, density target 0.865 g/cc) was added to the primary mixing vessel. The primary mixing vessel was then sealed and aggressively agitated until individual droplets of oil were no longer visible to the formulator. The product was then immediately dispensed into mist style pump bottles. The product showed complete turbidity and total suspension of mineral oil droplets for greater than 10 minutes and at times greater than 1 hour. Product separation may occur over several hours or days. Rehomogenization was achieved by several quick user-applied shakes or inversions. Product was applied to a user's hands by dispensing 2-3 pumps from the mist bottle onto one of the user's hands and spreading the liquid to the entirety of the user's hands. The product had clearly evident emollient properties. Oil could be felt on the skin after alcohol and water evaporated. The oil layer was slight and not greasy or heavy.

### Example 2

Exemplary hand sanitizer formulations were administered to panelists who were asked to common on their sensory effects compared to a control formulation.

In particular, on Day 1 of the analysis, panelists were given a control formulation including 75% (v/v) Alcohol (IPA) and 25% (v/v) DI Water. They were asked to apply 5 times throughout the day and record after each application whether they felt any residual effects. On Day 2 of the analysis, panelists were given samples of the exemplary hand sanitizer formulations and also asked to apply 5 times throughout the day and report on perceived effects after each application. They were asked for two key inputs: (1) Did they feel more or less residual effect than the formulation they applied on Day 1? (2) What comments about sensory effects came to mind in comparison to the formulation administered on Day 1? Four panelists participated in an analysis of three exemplary hand sanitizer formulations.

In a first analysis, an exemplary hand sanitizer formulation was selected from embodiments in which a low molecular weight alkane or mineral oil, such as saturated hydrocarbon alkane or low viscosity mineral oil, was completely dissolved in an alcohol solution. The exemplary hand sanitizer formulation given to the panelists on Day 2 of the analysis is identified above in Table 1, Formulation #2. 78% of the product usage experiences provided by the panelists stated that they felt more residual sensory effect as compared to the Day 1 control. Comments provided by the panelists included:
Left skin feeling “moisturized”
“Silky” feeling
Has a “residual feel”
Has more “play time”

In a second analysis, an exemplary hand sanitizer formulation was selected from embodiments having high emollient loading. The exemplary hand sanitizer formulation given to the panelists on Day 2 of the analysis is identified above in Table 2, Formulation #3. 100% of the product usage experiences provided by the panelists indicated that they felt more residual sensory effect as compared to the Day 1 control. Comments provided by the panelists included:
Has an “oil residue”
Feels like “moisturizer”
Feels like “lotion”
Hands feel “softer”

In a third analysis, an exemplary hand sanitizer formulation was selected from embodiments having density equalization. The exemplary hand sanitizer formulation given to the panelists on Day 2 of the analysis is identified above in Table 3, Formulation #1. 75% of the product usage experiences provided by the panelists indicated that they felt more residual sensory effect as compared to the Day 1 control. Comments provided by the panelists included:
Has a “lasting” effect
Hand feel “smoother”
Has an “oily feel”
Lasts “longer”

What is claimed is:

1. A hand sanitizer formulation, consisting essentially of:
an alcohol solution, wherein the alcohol solution comprises isopropanol at about 70% to about 90% of the hand sanitizer formulation by volume and distilled water at about 10% to about 29% of the hand sanitizer formulation by volume; and
a low molecular weight alkane or emollient at about 0.25% to about 5% of the hand sanitizer formulation by volume, wherein the alkane or emollient is fully solubilized in the alcohol solution, wherein the alkane or emollient is less than about 10% of the hand sanitizer formulation by weight, and wherein the hand sanitizer formulation is free of emulsifiers.

2. The hand sanitizer formulation of claim 1, wherein the alkane or emollient is a saturated hydrocarbon alkane having a carbon distribution centered between C12 to C17 average carbon chain length.

3. The hand sanitizer formulation of claim 1, wherein the saturated hydrocarbon alkane is C12-C17 alkane or C14-C22 alkane.

4. The hand sanitizer formulation of claim 1, wherein the alkane or emollient is a low viscosity mineral oil having a viscosity of less than 5 cSt at 40° C.

5. The hand sanitizer formulation of claim 1, wherein the alkane or emollient is petrolatum.

6. The hand sanitizer formulation of claim 1, further consisting essentially of glycerin at about 1.4% to about 1.5% by volume.

7. The hand sanitizer formulation of claim 1, further consisting essentially of fragrance at about 1% by volume.

8. The hand sanitizer formulation of claim 1, wherein the alcohol solution comprises isopropanol at about 79% to about 87% of the hand sanitizer formulation by volume and distilled water at about 10% to about 19% of the hand sanitizer formulation by volume, and wherein the low molecular weight alkane or emollient is at about 2% to about 3% of the hand sanitizer formulation by volume.

9. A hand sanitizer formulation, consisting essentially of:
isopropanol at about 70% by volume of the hand sanitizer formulation; and
one or more emollients at about 30% by volume of the hand sanitizer formulation, wherein the one or more emollients are more than 10% by weight of the hand sanitizer formulation, and wherein the hand sanitizer formulation is free of emulsifiers.

10. The hand sanitizer formulation of claim 9, wherein the one or more emollients comprise petrolatum, mineral oil, vegetable oil, vegetable wax, synthetic oil, synthetic wax, or combinations thereof.

11. The hand sanitizer formulation of claim 9, wherein the one or more emollients comprise sensory-enhanced petrolatum.

12. The hand sanitizer formulation of claim 9, further consisting essentially of fragrance at about 1% by volume.

13. A hand sanitizer formulation, consisting essentially of:
an alcohol solution comprising isopropanol or ethanol at about 60% to about 90% by volume of the hand sanitizer formulation and distilled water at about 10% to about 38% by volume of the hand sanitizer formulation; and
one or more emollients at about 2.3% to about 2.9% by volume of the hand sanitizer formulation, wherein the one or more emollients are less than 10% by weight of the hand sanitizer formulation, wherein there is no more than a 2% difference in density of the alcohol solution compared to density of the one or more emollients, and wherein the hand sanitizer formulation is free of emulsifiers.

14. The hand sanitizer formulation of claim 13, wherein the isopropanol or ethanol is at about 74% to about 77% by weight of the hand sanitizer formulation, wherein the distilled water is at about 20% to about 24% by weight of the hand sanitizer formulation, and wherein the one or more emollients is at about 2.4% to about 2.8% by weight of the hand sanitizer formulation.

15. The hand sanitizer formulation of claim 13, wherein the one or more emollients comprise mineral oil, alkanes, or petrolatum.

16. The hand sanitizer formulation of claim 13, wherein the one or more emollients comprise white mineral oil having a density of about 0.82 g/mL to about 0.89 g/mL at 20° C.

17. The hand sanitizer formulation of claim 13, further consisting essentially of fragrance at about 1% by volume.
